(19) United States
(12) Patent Application Publication
(10) Pub. No.: US 2025/0034655 A1
(43) Pub. Date: Jan. 30, 2025

(54) METHOD AND APPARATUS FOR SPERM SELECTION, SEPARATION, GENOMIC ANALYSIS, AND ASSISTED REPRODUCTION

(71) Applicant: Michael CHRISTENSEN, Brandenberg (DE)

(72) Inventor: Michael CHRISTENSEN, Brandenberg (DE)

(21) Appl. No.: 18/782,873

(22) Filed: Jul. 24, 2024

Related U.S. Application Data
(60) Provisional application No. 63/639,162, filed on Apr. 26, 2024.
(60) Provisional application No. 63/515,304, filed on Jul. 24, 2023.

(51) Int. Cl. C12Q 1/6888; C12N 5/071; G01N 15/01; G16H 10/40

(52) U.S. Cl. CPC C12Q 1/6888; C12N 5/0612; G01N 15/01; G16H 10/40

(57) ABSTRACT

Described herein are methods and systems for selecting and analyzing sperm cells. In particular, described herein are methods and systems for isolating bicephalic sperm cells and performing genomic analysis on the same while maintaining the viability of the sperm cell for implantation. Such systems and methods may be used in, for example, assisted reproduction and animal breeding applications.

### RELATED APPLICATIONS

This application claims the benefit of priority to U.S. Provisional Application No. 63/515,304 filed Jul. 24, 2023 and U.S. Provisional No. 63/639,162 filed Apr. 26, 2024, the entireties of which are incorporated herein by reference.

### FIELD OF THE DISCLOSURE

Various embodiments of the present disclosure relate generally to methods and apparatuses for selecting and separating spermatozoa, performing genomic analysis on the spermatozoa, and/or using these methods and apparatuses in aiding with assisted reproduction. More specifically, the embodiments of the present disclosure relate to leveraging bicephalic spermatozoa to augment polygenic preimplantation genetic testing.

### BACKGROUND

Example techniques discussed herein relate generally to assisted reproduction technologies (ART), and to a microfluidic device and method designed to select, separate, analyze, and/or use bicephalic spermatozoa for in-vitro fertilization (IVF) or animal breeding. While assisted reproductive technologies have significantly advanced, the selection of high-quality sperm remains challenging. Couples attempting to reproduce via IVF, for example, often have a very limited number of eggs. It would be advantageous to be able to pre-screen sperm to ensure lack of genetic disease, desired traits, etc., prior to fertilization. Existing techniques are limited by the ability to perform comprehensive genetic analyses on a single sperm without compromising viability. There is a need for an improved method and device to select high-quality sperm based on genetic content and viability.

The invention provides methods and devices that incorporate microfluidics, single-cell genomic analysis, cryopreservation, and IVF techniques for selecting high-quality bicephalic spermatozoa. The invention allows for the assessment of genetic material within sperm heads separately while preserving the associated sperm for potential use in IVF procedures.

### SUMMARY

Provided herein are methods for analyzing a bicephalic sperm cell. In some embodiments, the method may comprise: receiving a sample comprising sperm cells; isolating a bicephalic sperm cell from the sample, wherein the isolated bicephalic sperm cell comprises a first head and a second head; separating the first head of the isolated bicephalic sperm cell while retaining the second head on the isolated bicephalic sperm cell; performing, or causing to be performed, genomic analysis on the separated first head to produce genomic data; and associating the genomic data with the isolated bicephalic sperm cell. Isolating the bicephalic sperm cell may comprise separating the sperm cells using a centrifugal device and isolating the bicephalic sperm cell based on a predetermined weight or a predetermined weight range. The predetermined weight may be greater than or equal to about 20 pg. Isolating the bicephalic sperm cell may comprise separating the sperm cells using a flow cytometer and isolating the bicephalic sperm cell based on a predetermined optical property or a predetermined set of optical properties. The isolated bicephalic sperm cell may be viable after separating.

In some embodiments, associating the genomic data with the isolated bicephalic sperm cell may further comprise: determining a structural or morphological feature associated with the isolated bicephalic sperm cell; and based on the structural or morphological feature, determining a correspondence between the first head and the second head of the isolated bicephalic sperm cell. In some embodiments, associating the genomic data with the isolated bicephalic sperm cell may further comprise: generating a prediction of an association of the genomic data with the isolated bicephalic sperm cell based on recombination hotspots, linkage disequilibrium patterns, and/or population-specific genetic variations. In some embodiments, associating the genomic data with the isolated bicephalic sperm cell may be performed by a machine learning system trained with annotated data associating genomic data of both heads of bicephalic sperm cells, trained with high-resolution images of sperm heads and associated genomic data of the sperm heads to detect structural or morphological features, and/or trained to determine whether the first head and the second head derive from a single primary spermatocyte.

In some embodiments, separating the first head from the isolated bicephalic sperm cell may comprise sequential compartmentalization of the separated first head and the isolated bicephalic sperm cell. In some embodiments, the isolated bicephalic sperm cell may be selected to fertilize an oocyte based on a determination that the isolated bicephalic sperm cell negates an aneuploidy condition. In some aspects, upon determining that the isolated bicephalic sperm cell has been used to fertilize the oocyte to generate an embryo, the method may further comprise determining an inbreeding coefficient of the embryo via embryonic biopsy.

In some embodiments, associating the genomic data with the isolated bicephalic sperm cell may comprise utilizing DNA barcodes to identify and track the isolated bicephalic sperm cell and the cleaved head. In some embodiments, after separating the first head of the isolated bicephalic sperm cell, the method may further comprise cryopreserving the isolated bicephalic sperm cell while maintaining viability. In some embodiments, separating the first head of the isolated bicephalic sperm cell while retaining the second head on the isolated bicephalic sperm cell may comprise utilizing a microfluidics device. In some embodiments, performing, or causing to be performed, genomic analysis on the separated first head to produce genomic data may comprise performing probabilistic genomic prediction.

Further provided herein are systems for analyzing a bicephalic sperm cell. In some aspects, the system may comprise: a flow cytometer configured to isolate the bicephalic sperm cell based on a predetermined optical property or a predetermined set of optical properties; a microfluidics device integrated with the flow cytometer and configured to receive the isolated bicephalic sperm cell, wherein the microfluidics device is further configured to cleave a first head from the isolated bicephalic sperm cell while retaining the second head on the cell; a sequence analyzer configured to receive the cleaved first head from the microfluidics device and perform genomic analysis to produce genomic data; and a processor configured to associate the genomic data with the isolated sperm cell.

Further provided herein are methods for enhanced assisted reproduction. In some aspects, the method may comprise: receiving a sample comprising sperm cells; isolating a bicephalic sperm cell from the sample, wherein the isolated bicephalic sperm cell comprises a first head and a second head; separating the first head of the isolated bicephalic sperm cell while retaining the second head on the isolated bicephalic sperm cell; performing, or causing to be performed, genomic analysis on the separated first head to produce genomic data; associating the genomic data with the isolated bicephalic sperm cell; and selecting the isolated bicephalic sperm cell for in vitro fertilization, based on determining a probability that the isolated bicephalic sperm cell does not possess a chromosomal abnormality.

Further provided herein are methods for enhanced animal breeding. In some aspects, the method may comprise: receiving a sample comprising sperm cells; isolating a bicephalic sperm cell from the sample, wherein the isolated bicephalic sperm cell comprises a first head and a second head; separating the first head of the isolated bicephalic sperm cell while retaining the second head on the isolated bicephalic sperm cell; performing, or causing to be performed, genomic analysis on the separated first head to produce genomic data; associating the genomic data with the isolated bicephalic sperm cell; and selecting the isolated bicephalic sperm cell for in vitro fertilization, based on determining a probability that the isolated sperm cell possesses genetic material associated with desirable traits and a probability that the isolated sperm cell does not possess genetic material associated with undesirable traits.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 is a block diagram of a system for selecting, separating, and analyzing spermatozoa, according to techniques discussed herein.

FIG. 2a is an illustration depicting a bicephalic sperm cell, according to aspects described herein.

FIG. 2b is an illustration depicting a bicephalic sperm cell from which a first head has been cleaved, according to aspects described herein.

FIG. 3 is a block diagram depicting an exemplary method for obtaining and analyzing bicephalic sperm cells, according to aspects described herein.

FIG. 4 is a block diagram depicting an exemplary device for analyzing bicephalic sperm cell data, according to aspects described herein.

### DETAILED DESCRIPTION

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

Provided herein are systems, devices, and methods to select, separate, and analyze spermatozoa. Also described herein are methods of enhancing reproductive technologies with systems and devices for selecting, separating, and analyzing spermatozoa.

### Definitions

As used herein, the terms “spermatozoa”, “sperm”, and “sperm cells” refer to male gamete cells. A sperm cell can join an ovum (egg cell) to form a zygote deriving half of its genetic material from the sperm cell. Sperm cells can contain either an X chromosome or a Y chromosome. Sperm cells containing an X chromosome give rise to female zygotes (XX) upon joining with an ovum, while sperm cells containing a Y chromosome give rise to male zygotes (XY). Generally, a typical sperm cell comprises a head containing genetic material surrounded by an acrosome. Fused to the head is a midpiece which comprises mitochondria and powers movement of the tail. The tail comprises an axial filament that moves the sperm cell through a whip-like motion.

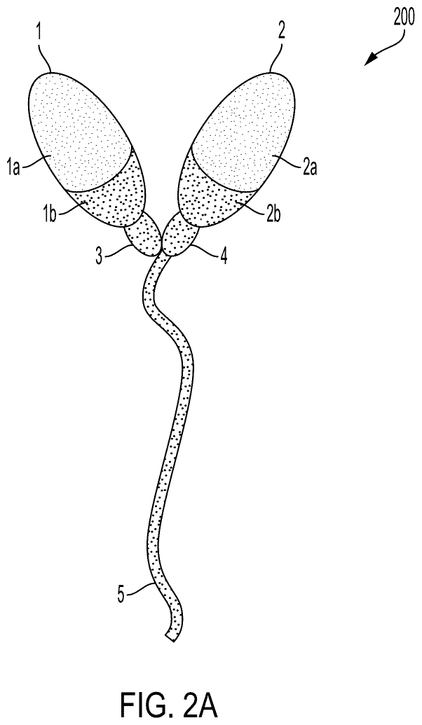

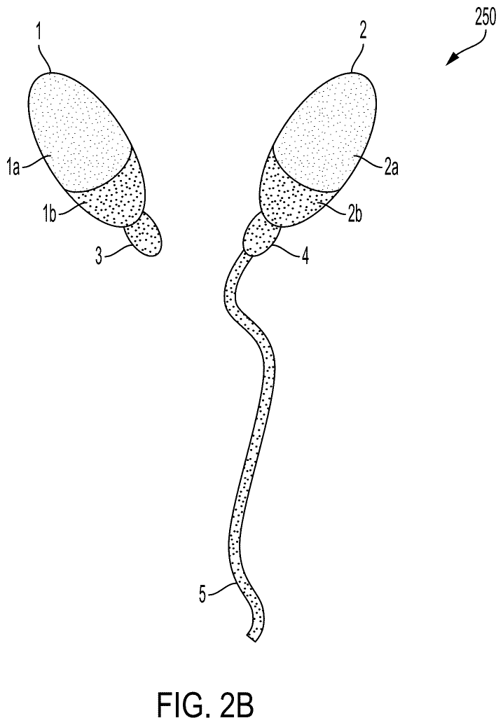

As used herein, the terms “bicephalic spermatozoa” or “bicephalic sperm cells” refer to sperm cells which have two heads fused to a single tail. Bicephalic sperm cells typically result from incomplete cellular division during spermatogenesis. The two heads of a bicephalic sperm cell may contain identical or different genetic material. Bicephalic sperm cells are rare, and estimated to make up less than 1% of sperm cells in a typical semen sample. However, the frequency may be higher in certain individuals or under specific conditions, such as genetic abnormalities or environmental exposures, and may be increased by certain interventions. For the purposes of illustration, FIG. 2a depicts an exemplary bicephalic sperm cell 200 comprising a first head 1, a second head 2, a first midpiece 3, a second midpiece 4, and a single, fused tail 5. First head 1 comprises a first acrosome 1a and a first postacrosomal cap 1b. Second head 2 comprises a second acrosome 2a and a second postacrosomal cap 2b. First midpiece 3 is connected to first head 1 and tail 5. Second midpiece 4 is connected to second head 2 and tail 5.

In some embodiments, incomplete cellular division during spermatogenesis may lead to sperm cells having three heads (tricephalic) or four heads (quadricephalic) fused to a single tail. In some embodiments, the methods and systems described herein may be adapted to isolate, analyze, and utilize rare polyheaded sperm structures, including but not limited to quadricephalic spermatozoa. Such structures may arise from successive cytokinetic failures during meiotic divisions of spermatogenesis, resulting in four or more sperm heads connected to a single tail structure.

As used herein, “deoxyribonucleic acid”, “DNA”, or “DNA molecules” refer to a nucleic acid molecule containing genetic information. As used herein, the term “genetic material” refers to molecules containing genetic information, such as DNA, ribonucleic acid (RNA), and chromosomes.

As used herein, the terms “viable” and “viability” refer to a sperm cell's structural integrity, motility, and capacity to fertilize an ovum (egg cell). These factors can be measured by semen analysis techniques. For example, structural integrity may be measured by determining membrane integrity, motility may be measured by observing motility patterns, and capacity to fertilize may be measured by observing morphology.

As used herein, the term “cytokinetic errors” refers to failures in the process of cytokinesis, the physical division of the cytoplasm at the end of cell division. In the context of spermatogenesis, incomplete cytokinesis can lead to conjoined sperm.

As used herein, the term “pseudo-bicephaly” refers to the fusion of two separate sperm, often at the tail region, giving the appearance of a bicephalic structure.

As used herein, the term “microfluidics” refers to a system and/or biochemical technique designed with a network of narrow (micro) channels to manipulate tiny amounts of fluid flowing through the channels that may be 10-1000 micrometers in size. The microchannel network allows for precise control over the small amount of fluid and thereby allows for the performance of a number of biochemical techniques that would otherwise require large amounts of fluids and/or physical space.

As used herein, the terms “droplet microfluidics” or “droplet-based microfluidics” refer to a biochemical technique of precisely controlling the formation of droplets from immiscible fluids. The droplets can be used to separate and/or encapsulate particles or cells. The droplets can further serve as reaction sites to perform independent reactions on the separated cells.

As used herein, the term “flow cytometry” refers to a biochemical technique used to detect and measure physical and/or chemical characteristics of a population of cells. In general, flow cytometry may be performed by suspending cells in a fluid and injecting the suspension into a flow cytometer. In the flow cytometer, cells flow in single file through a laser beam, and scattering of light from the laser beam is measured, logged, and evaluated as optical properties of the cells. The cells may be stained and labeled with a fluorescent dye prior to injection into the flow cytometer.

As used herein, the term “density gradient centrifugation” refers to a centrifuge-based technique used to separate cells or particles based on size, shape, weight, or density. The technique uses several media of varying densities within a single container and applies a centrifugal force to the container, causing cells to migrate through the various media at different rates based on their differing properties. Cells of different properties may concentrate in different media at different positions within the container.

As used herein, “microfiltration” and “nanofiltration” refer to a physical filtration technique utilizing a porous membrane to remove contaminants from a fluid flowing through the porous membrane. Microfiltration utilizes a membrane with pores approximately 0.1 μm to 10 μm in diameter, whereas nanofiltration utilizes a membrane with pores approximately 1 nm to 10 nm in diameter.

As used herein, the term “genomic analysis” refers to biochemical techniques for analyzing and recording the genetic sequences contained in a genome of a target cell. As used herein, “probabilistic genomic prediction” refers to utilizing computational models to infer genetic markers associated with phenotypic traits and heritable diseases, as described in further detail herein.

As used herein, the term “DNA barcoding” refers to a biochemical technique of inserting short (e.g., about 10-20 nucleotides in length) DNA molecules, having unique nucleic acid sequences, into the genome of a target cell for identification and tracking of the target cell. These short DNA molecules may be referred to as “DNA barcodes,” and may be designed with specific sequences for polymerase chain reaction (PCR) amplification that can be decoded during genomic analysis of the target cell. DNA barcodes may be inserted into the genome of a target cell by, for example, utilizing droplet microfluidics or surface conjugation.

As used herein, the term “cryopreservation” refers to a biochemical technique of freezing biological material, such as cells, in a manner designed to preserve the viability of the material over time.

As used herein, the terms “assisted reproduction technologies” or “ART” refer to technologies and treatments designed to assist in sexual reproduction, such as in vitro fertilization, gamete intrafallopian transfer, and intrauterine insemination.

### Methods for Isolating and Analyzing Bicephalic Sperm Cells

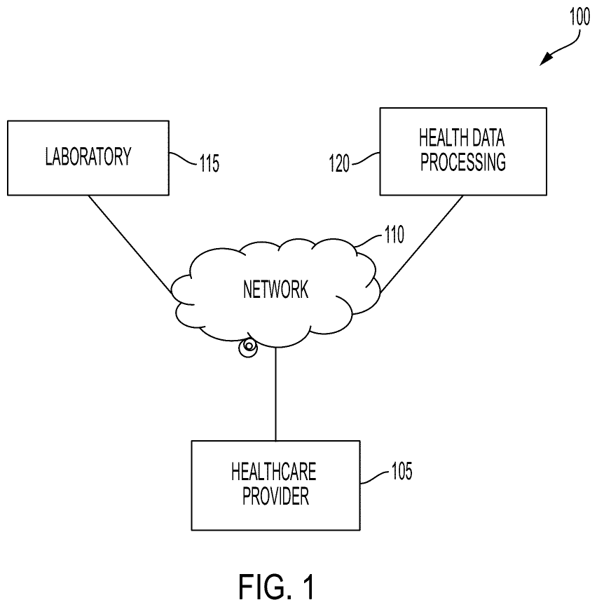

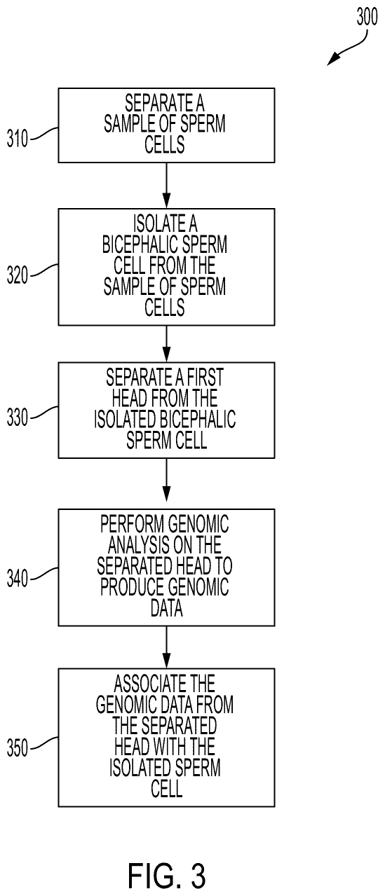

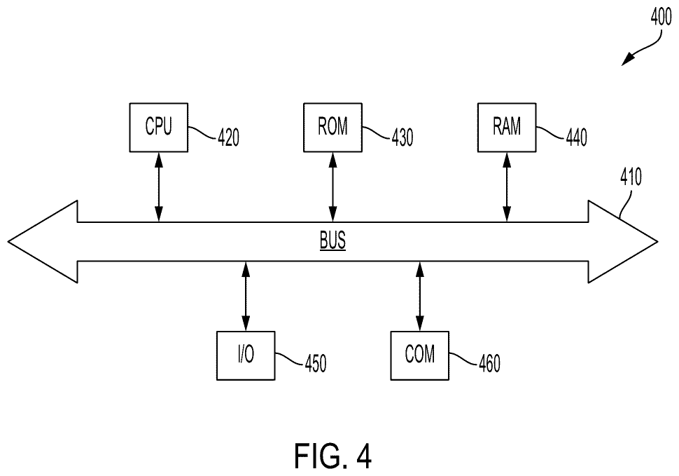

FIG. 1 is a block diagram of a system for selecting, separating, and analyzing spermatozoa, in particular bicephalic sperm, according to techniques discussed herein. A couple seeking IVF may engage a healthcare provider, for example a fertility doctor. The healthcare provider 105 may collect a semen sample from the male, and may analyze it internally and/or engage a laboratory 115 to perform further analysis. The healthcare provider 105 may utilize devices to communicate across network 110 with devices at laboratory 115. The network communication devices may comprise computers, for example those discussed in FIG. 4. The network 110 may comprise the Internet, a Wi-Fi network, local area network (LAN), intranet, wireless, Bluetooth, Near Field Communication (NFC), and/or any wired or wireless data connection, or any combination thereof. While the laboratory, healthcare provider, and health data processors may be depicted as separate herein, they also may be the same entity. The healthcare provider 105 may provide data related to the semen sample and/or spermatozoa contained therein, information about the male or female patients, demographic information, family history, medical history or information, etc. to the laboratory 115. The laboratory 115 may return test results pertaining to the semen/sperm, and may perform techniques discussed in relation to FIGS. 2a, 2b, and 3. Health data processing 120 may provide further data analysis, for example processing data from laboratory 115 to generate a prediction of genetic traits of individual sperm.

As described above, a bicephalic sperm cell comprises two heads fused to one tail. The methods described herein involve cleaving or otherwise separating one head from an isolated bicephalic sperm cell while retaining the other head on the sperm cell. For the purposes of illustration only, FIG. 2b illustrates a bicephalic sperm cell after cleavage 250. First head 1, comprising first acrosome 1a and first post-acrosomal cap 1b, and first midpiece 3 are separate. Sperm cell 200 comprises only second head 2 comprising second acrosome 2a and second post-acrosomal cap 2b, second midpiece 4, and tail 5.

In some aspects, the method comprises isolating a bicephalic sperm cell from a sample of sperm cells, cleaving or otherwise separating a first head from the isolated sperm cell while retaining a second head on the isolated sperm cell, performing genomic analysis on, for example, the cleaved first head, and associating the genomic analysis from the cleaved head with the isolated sperm cell.

FIG. 3 depicts an exemplary embodiment of the methods described herein. At step 310, a sample of sperm cells (e.g., a semen sample containing sperm cells) may be received. At step 320, a bicephalic sperm cell may be isolated from the sample of sperm cells. Isolating a bicephalic sperm cell may be performed using a variety of known techniques, including, but not limited to, flow cytometry, density gradient centrifugation, microfluidic sorting, or any combination thereof. The techniques used must maximize the yield of viable bicephalic sperm cells from the sample while simultaneously excluding potentially damaged sperm cells.

In some embodiments, isolating a bicephalic sperm cell comprises separating the sample of sperm cells by flow cytometry, as described herein, and identifying a bicephalic sperm cell based on its unique optical properties relative to normal, single-headed sperm cells. In some embodiments, flow cytometry may be used to isolate and analyze a bicephalic sperm cell based on its unique light scattering properties due to its atypical morphology. In some embodiments, flow cytometry may be combined with fluorescent labeling techniques to provide information about DNA content, viability, and other biochemical characteristics of the bicephalic sperm cells, allowing for high-throughput screening and sorting of sperm cells, potentially increasing the efficiency of identifying and isolating bicephalic sperm for further analysis.

In some embodiments, isolating a bicephalic sperm cell comprises separating the sample of sperm cells by weight and identifying a bicephalic sperm cell based on its unique weight relative to normal, single-headed sperm cells. Typical sperm cells may have an average volume of about 17 cubic microns (e.g., 17 μm³) and a weight of less than 20 picograms (i.e., 20 pg). See, for example, Laufer et al., “Volume and shape of normal human spermatozoa”, Fertility and Sterility, 1977, vol. 8, no. 4, pages 456-458, the contents of which are incorporated herein by reference. By contrast, atypical sperm cells may have atypical weights. For example, bicephalic sperm cells may typically have a weight slightly greater than the weight of typical sperm cells, due to the added weight of the second head. In some embodiments, a bicephalic sperm cell may have a weight greater than or equal to about 20 pg. For example, a bicephalic sperm cell may have a weight of about 20 pg to about 50 pg, about 20 pg to about 40 pg, about 20 pg to about 30 pg, about 20 pg to about 25 pg, about 20 pg to about 22 pg, or about 20 pg to about 21 pg.

In some embodiments, separating a sample of sperm cells by weight comprises performing density gradient centrifugation. Density gradient centrifugation utilizes centrifugal forces to separate components in a solution by their respective weights. In this manner, density gradient centrifugation allows for the collection of relatively low amounts of certain components. In some embodiments, performing density gradient centrifugation on a sample of sperm cells found in, for example, a sample in a container results in a concentration of bicephalic sperm cells at one end of the container.

In some embodiments, isolating a bicephalic sperm cell comprises determining whether a sperm cell having a weight associated with a bicephalic sperm cell, as described herein, is bicephalic or is not bicephalic. A sperm cell having a weight associated with a bicephalic sperm cell may be a non-bicephalic sperm cell comprising other, atypical features causing an increased weight of the cell. For example, a non-bicephalic sperm cell may have a weight similar to or the same as a weight of a bicephalic sperm cell due to cytokinetic errors or pseudo-bicephaly. In some embodiments, determining whether a sperm cell having a weight associated with a bicephalic sperm cell is bicephalic or is not bicephalic comprises identifying other features unique to bicephalic sperm cells. In some embodiments, a feature unique to bicephalic sperm cells may be a particular morphology, which may be identified using high-resolution imaging techniques.

At step 330, one head of the bicephalic sperm cell may be cleaved or otherwise separated, while the other head is retained on the sperm cell. In some embodiments, cleaving a first head from an isolated bicephalic sperm cell retains the second head on the isolated sperm cell. In some embodiments, the isolated sperm cell retaining the second head remains viable after cleaving.

In some embodiments, cleaving a first head from an isolated bicephalic sperm cell comprises mechanically or chemically separating the first head from the remainder of the isolated bicephalic sperm cell. In some embodiments, mechanically separation comprises using a device configured for mechanical separation. In some embodiments, devices for mechanical separation may be produced using microfabrication techniques, such as soft lithography. In some embodiments, devices for mechanical separation may incorporate fluid flow control components, such as syringe pumps or pressure systems, for accurately positioning and shearing the first head from the isolated bicephalic sperm cell. In some embodiments, the device for mechanical separation comprises a microfluidic guillotine at a Y-shaped intersection configured to shear the first head from the remainder of the isolated bicephalic sperm cell. For example, an isolated bicephalic sperm cell may be guided into a microfluidic device containing a narrow channel or constriction configured to apply a precise mechanical force to the sperm sufficient to sever the first head from the sperm cell. See, for example, Blauch et al., “Microfluidic guillotine for single-cell wound repair studies”, PNAS, 2017, vol. 114, no. 28, pages 7283-7288, the contents of which are hereby incorporated by reference in their entirety. In some embodiments, the device for mechanical separation comprises a polydimethylsiloxane (PDMS) or other polymer-based device.

At step 340, genomic analysis may be performed on the cleaved head to produce genomic data. In some embodiments, performing genomic analysis on a cleaved head comprises sequencing and genetic analysis techniques. Techniques for genomic analysis include, but are not limited to, genotyping, high-throughput single-cell sequencing, or any other genomic analysis technology.

At step 350, the genomic data produced from genomic analysis of the cleaved head may be associated with the isolated sperm cell from which the cleaved head was derived. For example, the two heads may be genetically identical, or otherwise contain a larger than random degree of similarity. The two heads may be genetically complementary in a predetermined way, such that sequencing one head allows derivation of genetic information about the other. Further, structural or morphological features of the cleaved sperm head, which may be determined via chemical staining, high-resolution imaging, or other chemical means, may predict the correspondence of the genetic material between the two sperm heads, as will be discussed further below.

In some embodiments, associating the genomic data from the cleaved first head with the remainder of the isolated bicephalic sperm cell comprises sequential compartmentalization utilizing a microfluidic device having parallel channels, as described herein. After cleavage, each sperm head pair may be encapsulated in a single droplet, then split into two linked droplets containing the cleaved head and the remaining cell, respectively. Droplet pairs may move through the parallel channels, maintaining their association throughout analysis. In some embodiments, associating the genomic data comprises utilizing cellular identification and tracking techniques. In some embodiments, the cellular identification and tracking technique comprises DNA barcoding, such as exposing the isolated bicephalic sperm cell to a unique DNA molecule configured to insert into the cell's genome. In some embodiments, the cellular identification and tracking technique comprises dying the droplets containing the cleaved first head and the remainder of the isolated bicephalic sperm cell.

In some embodiments, associating the genomic analysis from the cleaved head with isolated sperm cell comprises storing the isolated sperm cell in a known location, such as a labeled or identifiable compartment. In some embodiments, storing the isolated sperm cell comprises conditions maintaining viability of the isolated sperm cell, such as in cold storage or with chemical stabilization.

As described above, depending on biological mechanisms underlying bicephalicism, a bicephalic sperm cell may comprise two heads having identical genetic material or different genetic material. In some embodiments, methods described herein comprise determining identity of genetic material between two heads of a bicephalic sperm cell. In some aspects, determining identity of genetic material between two heads of a bicephalic sperm cell comprises selecting a second bicephalic sperm cell, performing genomic analysis on both heads of the second bicephalic sperm cell, and using the genomic analysis to identify a pattern or base rate specific to the individual from whom both bicephalic sperm cells were obtained using statistical techniques and algorithms, as described herein. Based on the number of bicephalic sperm analyzed, the confidence of the prediction of likelihood of identical genetic material between two heads gradually increases. Alternatively, a machine learning system may be trained on bicephalic sperm genetic correspondence data, which may be annotated, and genetic analyses from an individual patient may increase the confidence of the machine learning system. The healthcare provider might act upon the predictions of the machine learning system when the confidence level exceeds a predetermined threshold.

In some aspects, a method for determining identity of genetic material between two heads of a bicephalic sperm cell may comprise selecting a second bicephalic sperm cell, and identifying structural or morphological features associated with identical genetic material between the two heads. In some embodiments, identifying the structural or morphological feature comprises using techniques including, but not limited to, high-resolution imaging and image processing, chemical tests, flow cytometry, machine learning, and any combination thereof. For example, high-resolution imaging techniques, such as electron microscopy or super-resolution fluorescence microscopy, may be used to produce high-resolution stained/virtually stained images to be processed and analyzed by a machine learning algorithm trained to detect subtle structural or morphological differences and identify a unique structural or morphological feature. As another example, flow cytometry may be used to produce data on optical properties to be processed and analyzed by a machine learning algorithm to identify structural or morphological features. As another example, a machine learning algorithm could be used to identify molecular markers specific to different stages of spermatogenesis or epigenetic modification.

Identical genetic material between the two heads of a bicephalic sperm cell is not crucial for the effectiveness of the present disclosure. For example, genomic analysis of one head might allow for probabilistic genomic prediction of the other. Understanding of recombination patterns (e.g., recombination hotspots and chromatid/crossover interference) might permit sperm selection for maximum variance, enhancing potential benefits after fertilization as part of embryo selection. In the context of the present disclosure, probabilistic genomic prediction refers to the idea of estimating genetic content of one sperm head based on the information obtained from genomic analysis of its paired head. This approach relies on an assumption that two heads originate from the same primary spermatocyte and thus share a significant portion of their genetic material. The machine learning model discussed above may further be trained to determine a likelihood that the two heads originate from the same primary spermatocyte. For example, if the sequenced sperm head contains a particular allele at a given locus, the probability of the other head having the same allele would depend on the proximity of that locus to a recombination site. Loci closer to recombination points would have a higher chance of being different between the two heads, while those further away would be more likely to be identical. The analysis might also be restricted to cases in which cytokinetic errors occur in both secondary spermatocytes, resulting in two pairs of bicephalic sperm, a relatively uncommon circumstance, yet possible due to the vast number of sperm in a semen sample. If bicephalic sperm is the result of incomplete cytokinesis during Meiosis 2, yielding non-identical DNA, sequencing three sperms from the same primary spermatocyte will enable precise identification of the genetic material in the fourth.

In some embodiments, probabilistic genomic prediction may involve the following steps: sequencing the genome of a first (cleaved) sperm head to obtain a high-resolution genetic map; identifying recombination events and crossover points that occurred during Meiosis I, when the primary spermatocyte divided into two secondary spermatocytes; and utilizing the knowledge of recombination patterns and frequencies in the human genome, as well as models of recombination and chromatid interference (e.g., gamma model, beam-film model, or Hidden Markov model as described herein) to generate a prediction of the likely genetic composition of a second (retained) sperm head.

In some embodiments, computational models and algorithms are used to generate these predictions, taking into account factors such as recombination hotspots, crossover interference, linkage disequilibrium patterns, and population-specific genetic variations. In some embodiments, machine learning algorithms trained on large datasets of known sperm genomes and their recombination profiles are utilized to enhance the accuracy of these predictions. These steps may be performed by any entity of FIG. 1, including health data processing 120.

In some embodiments, the method further comprises, prior to cleaving, encapsulating the isolated bicephalic sperm cell in a fluidic or aqueous environment. In some embodiments, the fluidic or aqueous environment comprises an encapsulation medium. In some embodiments, the encapsulation medium comprises nutrients or other factors that promote cell survival or viability. In some embodiments, encapsulating the isolated bicephalic sperm comprises utilizing droplet microfluidics, as described herein.

In some embodiments, the method further comprises, after cleaving and separating, compartmentalizing the sheared first head of the isolated bicephalic sperm cell and the remainder of the isolated bicephalic sperm cell. In some embodiments, compartmentalizing comprises sequential compartmentalization. In some embodiments, compartmentalizing the remainder of the isolated bicephalic sperm cell comprises maintaining the cell in conditions to promote cell survival or viability. In some embodiments, compartmentalizing the remainder of the isolated bicephalic sperm cell comprises cryopreserving the cell for future use, as described herein. In some embodiments, compartmentalizing comprises encapsulating the remainder of the isolated bicephalic sperm cell in a fluidic or aqueous environment. In some embodiments, the fluidic or aqueous environment comprises an encapsulation medium. In some embodiments, the encapsulation medium comprises nutrients or other factors that promote cell survival or viability. In some embodiments, encapsulating the isolated bicephalic sperm comprises utilizing droplet microfluidics as described herein.

In some embodiments, the method further comprises removing contaminants before, during, or after cleaving. Examples of contaminants include, but are not limited to, cellular debris, particulate debris, residual seminal plasma, immature sperm cells, white blood cells, epithelial cells, or any combination thereof. In some embodiments, removing contaminants comprises utilizing techniques including, but not limited to, microfiltration, nanofiltration dielectrophoresis, and acoustofluidics.

### Systems and Devices for Isolating and Analyzing Bicephalic Sperm Cells

Disclosed herein are systems and devices for isolating and analyzing a bicephalic sperm cell. In some aspects, the system comprises a means for separating sperm cells in a sample, a means for extracting and/or isolating a bicephalic sperm cell from the separated sample, a means for cleaving a first head from the isolated bicephalic sperm cell while retaining a second head on the isolated sperm cell, a means for performing genomic analysis on the cleaved first head, and/or a means for associating data from the genomic analysis with the isolated sperm cell.

In some embodiments, the means for separating sperm cells in a sample comprises a flow cytometer configured for isolating and analyzing bicephalic sperm cells. The flow cytometer may include: a fluidics system for aligning sperm cells in a fluid stream; one or more lasers for illuminating the sperm cells; optical filters and detectors for measuring forward scatter (FSC), side scatter (SSC), and fluorescence emissions; a computer system for data analysis; and a cell sorting mechanism for separating bicephalic sperm cells. In some embodiments, the flow cytometer may be configured to identify bicephalic sperm cells based on their light scattering properties. Bicephalic sperm cells may produce distinct FSC and SSC signals compared to typical sperm cells due to their increased size and complexity. In some embodiments, the flow cytometer may include fluorescence detection capabilities. These may comprise: DNA staining dyes to quantify DNA content; viability dyes to assess sperm cell viability; fluorescently labeled antibodies for detecting specific biomarkers; or any combination thereof. In some embodiments, the flow cytometer may be integrated with a microfluidics device as described herein, facilitating the transition from isolation to analysis and cleaving of bicephalic sperm cells. In some embodiments, the flow cytometer may employ a multi-parameter analysis approach to identify bicephalic sperm cells. This approach may include: analysis of FSC and SSC signal ratios; pulse shape analysis; fluorescence analysis to confirm the presence of two distinct nuclei; or any combination thereof.

In some embodiments, the means for separating sperm cells in a sample comprises a centrifugal device. In some embodiments, the centrifugal device is a density gradient centrifuge as described herein.

In some embodiments, the means for cleaving a first head from the isolated bicephalic sperm cell while retaining a second head on the isolated sperm cell comprises a microfluidics device or system. The microfluidics device may be configured to generate droplets as described herein. In some techniques, the microfluidics device comprises microvalves or microwells.

In some embodiments, the means for performing genomic analysis on the cleaved first head comprises a sequence analyzer. In some embodiments, the sequence analyzer is configured for high-throughput single-cell sequencing.

In some embodiments, the means for associating data from the genomic analysis with the isolated sperm cell comprises a processor, such as a computer processor.

In some embodiments, the system further comprises a means for detecting structural or morphological features indicative of genetic identity between two heads of an isolated bicephalic sperm cell, which may correspond to those techniques discussed above.

In some embodiments, the system further comprises software configured to improve bicephalic sperm identification based on operator feedback and genomic analysis results.

### Assisted Reproduction Technologies

The methods and systems described herein may be used to enhance the efficacy of assisted reproduction technologies. For example, genomic data produced from a first head cleaved from an isolated bicephalic sperm cell can serve as a basis for selecting high-quality, viable sperm cells for use in, for example, assisted reproduction. Criteria for selection of high-quality sperm cells include, but are not limited to: overall DNA integrity, DNA fragmentation levels, polygenic scores, genetic markers of interest, single genes or mutations related to disorders, chromosomal aneuploidy, structural abnormalities, microdeletions impacting fertility, copy number variations, and epigenetic modifications (such as DNA methylation or histone retention). Once a selection is made based on the genomic data from the cleaved first head, the corresponding isolated sperm cell (now no longer bicephalic) may be utilized in assisted reproduction procedures, such as in vitro fertilization.

As another example, the methods and systems described herein may be used to correct chromosomal deviations. A bicephalic sperm cell typically contains a total of 46 chromosomes distributed between two heads. However, deviations from the standard 23 haploid chromosomes per sperm head can occur, offering a unique opportunity for aneuploidy correction. For example, if one sperm head presents an aneuploidy condition such as monosomy or trisomy for any number of specific chromosomes (e.g., zero or two copies of chromosome 1), it may be inferred that the complementary sperm head will compensate with an adjusted number of the same chromosomes in the specific chromosome. High-resolution imagining and staining of the bicephalic sperm may help in identifying bicephalic sperm that contain an uneven set of chromosomes between the two heads. This knowledge may be instrumental when used in conjunction with oocytes exhibiting contrary chromosomal imbalances. Specifically, a sperm head with a duplication of a particular chromosome can be strategically paired with an oocyte deficient in that chromosome to facilitate the conception of an euploid embryo, thereby restoring a normal chromosomal total of 46. Conversely, a sperm head with a deficiency in a certain chromosome can complement an oocyte with an excess of that chromosome. The methods and systems described herein further allow for the rescue of an egg cell (oocyte, ovum) with one or more chromosomal abnormalities by identifying a sperm cell that contains multiple complementary aneuploidies. The chromosomal status of the oocyte may be ascertained via a polar body biopsy and comprehensive genomic sequencing of the egg donor's saliva/blood/any number of somatic cells. This allows precise pairing of sperm and oocyte based on complementary chromosomal profiles, enhancing the likelihood of correcting chromosomal discrepancies in the oocyte.

Post-fertilization, the success of the aneuploidy correction and overall chromosomal integrity of the embryo may be verified through embryonic biopsy followed by whole-genome sequencing of the biopsy. Verifying the embryo may comprise confirming that the embryo is euploid as well as assessing whether any potential inbreeding effects/deleterious pathogenic recessive conditions have been introduced. Since aneuploidy correction implies that at least one chromosome pair originates entirely from one of the parent's alone it may be crucial to test for any inbreeding/recessive variant problems. These assessments may involve meticulous analysis of the inbreeding coefficient such as checking for runs of homozygosity and comparison with established databases for recessive mutations, alongside comparisons with known population inbreeding rates to estimate the safety of the intervention. The methods and systems described herein significantly advances the capabilities of ensuring the maximum number of oocytes may be fertilized by enabling targeted interventions for chromosomal correction and thus, contributing profoundly to the success rates of assisted reproductive technologies.

### Animal Breeding

The methods and systems described herein are not limited to use with human reproduction. For example, the methods and systems described herein may be used in animal breeding programs, where enhancement of genetic selection procedures can lead to improved outcomes.

In some embodiments, the methods and systems disclosed herein may be advantageously employed for enhancing genetic selection in animal breeding programs. Such applications may include, but are not limited to, accelerating genetic improvement in livestock, aquaculture, and conservation efforts for endangered species. The disclosed techniques may enable more precise selection of desirable traits, including but not limited to, disease resistance, growth rate, and production characteristics (e.g., meat quality, milk yield), while concurrently maintaining genetic diversity. The capability to analyze the genome of one sperm head while preserving the viability of the other proves particularly valuable in scenarios where genetic material is limited or highly valuable, such as in breeding programs for rare or endangered animals. Furthermore, the methods and systems disclosed herein may reduce the number of breeding animals required, thereby potentially increasing the efficiency of breeding operations.

In some embodiments, the disclosed methods may be applied to sperm cells obtained through testicular biopsy, which may offer a valuable alternative for obtaining bicephalic sperm cells. Testicular tissue samples, procured through minimally invasive biopsy procedures, may yield a higher proportion of bicephalic sperm compared to ejaculated samples. Without intending to be bound by any particular theory, this increased yield is hypothesized to result from reduced selection pressure within the testes, allowing for the preservation of sperm cells that might otherwise be eliminated during the maturation and ejaculation processes. The biopsy approach may be especially beneficial for valuable breeding animals, as it allows for repeated sampling without the need for frequent semen collection, which can be stressful or impractical for some species. Moreover, testicular biopsies can be combined with in vitro spermatogenesis techniques to potentially increase the yield of bicephalic sperm cells. Specialized protocols may be necessary for the preservation and analysis of sperm obtained through biopsy, as these cells may differ in their maturation stage compared to ejaculated sperm. The integration of testicular biopsy techniques with the bicephalic sperm analysis methods described herein has the potential to significantly enhance the efficacy of genetic selection in animal breeding programs, particularly for species where traditional semen collection is challenging or where genetic material is scarce and highly valuable.

The present disclosure contemplates modifications to microfluidic devices described herein to accommodate sperm structures of varying sizes and morphologies. Such modifications may include, but are not limited to, adjustable channel dimensions, adaptive flow rates, and specialized sorting mechanisms capable of distinguishing and isolating polyheaded sperm structures from typical sperm populations.

In some embodiments embodiment, genomic analysis methods disclosed herein may be expanded to compare and contrast the genetic content of multiple heads from the same polyheaded sperm structure. This analysis may comprise: a) sequentially separating individual heads from the polyheaded structure; b) performing whole-genome amplification and sequencing on each separated head; c) utilizing machine learning algorithms to analyze the genetic content of each head in relation to the others; and d) generating predictive models of genetic recombination and segregation patterns based on the analyzed data.

### Computer-Implemented Systems and Devices

FIG. 4 illustrates an example computer-implemented system or device 400 that may execute techniques presented herein, and may correspond to devices shown in FIG. 1, such as devices associated with laboratory device(s) 115, health care provider device(s) 105, health data processing device(s) 120, and/or network 110. Device 400 may include a central processing unit (CPU) 420. CPU 420 may be any type of processor device including, for example, any type of special purpose or a general-purpose microprocessor device. As will be appreciated by persons skilled in the relevant art, CPU 420 also may be a single processor in a multi-core/multiprocessor system, such system operating alone, or in a cluster of computing devices operating in a cluster or server farm. CPU 420 may be connected to a data communication infrastructure 410, for example a bus, message queue, network, or multi-core message-passing scheme.

Device 400 may also include a main memory 440, for example, random access memory (RAM), and also may include a secondary memory 430. Secondary memory 430, e.g. a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 430 may include similar means for allowing computer programs or other instructions to be loaded into device 400. Examples of such means may include a program cartridge and cartridge interface (such as that found in video game devices), a removable memory chip (such as an EPROM or PROM) and associated socket, and other removable storage units and interfaces, which allow software and data to be transferred from a removable storage unit to device 400.

Device 400 also may include a communications interface (COM) 460. Communications interface 460 allows software and data to be transferred between device 400 and external devices. Communications interface 460 may include a modem, a network interface (such as an Ethernet card), a communications port, a PCMCIA slot and card, or the like. Software and data transferred via communications interface 460 may be in the form of signals, which may be electronic, electromagnetic, optical or other signals capable of being received by communications interface 460. These signals may be provided to communications interface 460 via a communications path of device 400, which may be implemented using, for example, wire or cable, fiber optics, a phone line, a cellular phone link, an RF link or other communications channels.

The hardware elements, operating systems, and programming languages of such equipment are conventional in nature, and it is presumed that those skilled in the art are adequately familiar therewith. Device 400 may also include input and output ports 450 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Throughout this disclosure, references to components or modules generally refer to items that logically may be grouped together to perform a function or group of related functions. Like reference numerals are generally intended to refer to the same or similar components. Components and/or modules may be implemented in software, hardware, or a combination of software and/or hardware.

The tools, modules, and/or functions described above may be performed by one or more processors. “Storage” type media may include any or all of the tangible memory of the computers, processors or the like, or associated modules thereof, such as various semiconductor memories, tape drives, disk drives and the like, which may provide non-transitory storage at any time for software programming.

Software may be communicated through the Internet, a cloud service provider, or other telecommunication networks. For example, communications may enable loading software from one computer or processor into another. As used herein, unless restricted to non-transitory, tangible “storage” media, terms such as computer or machine “readable medium” refer to any medium that participates in providing instructions to a processor for execution.

### Example 1 (Prophetic)

A sample of sperm cells obtained from a healthy human male donor is microfiltered to remove contaminants and centrifuged in a density gradient centrifuge to separate sperm cells based on weight. Sperm cells having a weight in the range of known weights for bicephalic sperm cells are extracted and isolated. Each bicephalic sperm cell is encapsulated using droplet microfluidics, with each droplet being dyed a unique color. Encapsulated bicephalic sperm cells are guided into a microfluidics device containing a microfluidic guillotine at a Y-shaped intersection which mechanically separates one head from each bicephalic sperm cell. Isolated sperm cells are stored via cryopreservation. Separated heads are sequenced in a sequence analyzer, and genomic analysis is associated with a cryopreserved sperm cell based on matching droplet dye color.

### Example 2 (Prophetic)

A semen sample is obtained from a male patient seeking fertility treatment. The sample is processed through a flow cytometer specially configured to identify bicephalic sperm cells based on their unique light scattering properties and DNA content. Identified bicephalic sperm cells are isolated and individually encapsulated in microfluidic droplets, each labeled with a unique DNA barcode for tracking. The encapsulated bicephalic sperm cells are then guided through a microfluidic device with a Y-shaped intersection. At this intersection, a precisely controlled laser pulse is used to separate one head from each bicephalic sperm cell. The separated heads are immediately directed to a high-throughput single-cell sequencing system for genomic analysis. Meanwhile, the remaining sperm cells (now with a single head) are cryopreserved. The genomic data from the sequenced heads is analyzed using machine learning algorithms trained on a database of known bicephalic sperm genetic profiles. This analysis generates predictions about the genetic content of the corresponding cryopreserved sperm cells. Based on this analysis, a specific cryopreserved sperm cell is selected for in vitro fertilization. The selection is made to complement the genetic profile of a donor egg, with the aim of minimizing genetic disease risk and optimizing desired traits. After fertilization and embryo development, a small embryonic biopsy is performed to confirm the genetic predictions and assess the overall health of the embryo before implantation.

### Example 3 (Prophetic)

A herd of prize dairy cattle is selected for genetic improvement using the methods described herein. Testicular biopsies are performed on the top three bulls in the herd, chosen based on their superior milk production traits. The biopsied tissue samples are processed to isolate sperm cells, with particular attention given to identifying bicephalic sperm. The samples are filtered using a microfluidic device equipped with size-exclusion channels optimized for bovine sperm dimensions.

Isolated bicephalic sperm cells are individually encapsulated in hydrogel microspheres, each containing a unique DNA barcode for tracking. Next, the encapsulated sperm cells are guided through a microfluidic sorting device that utilizes laser-induced shockwaves to separate one head from each bicephalic sperm cell. The separated heads are immediately subjected to whole-genome amplification followed by high-throughput sequencing.

Concurrently, the remaining sperm cells (now with a single head) are cryopreserved in individual containers, each labeled with a barcode corresponding to its sequenced counterpart. The genomic data from the sequenced heads is analyzed using a machine learning algorithm trained on a database of bovine genetic profiles associated with superior milk production traits, including protein content, fat percentage, and overall yield.

Based on this analysis, specific cryopreserved sperm cells are selected for in vitro fertilization with oocytes collected from the herd's top-producing cows. The selection is made to maximize the probability of offspring inheriting desired milk production traits while minimizing the risk of genetic disorders common in dairy cattle.

After fertilization and embryo development, a small embryonic biopsy is performed on each embryo to confirm the genetic predictions and assess overall health before implantation into surrogate cows. The resulting calves are monitored throughout their development, with particular attention to their milk production traits upon maturation.

This process is repeated over several breeding cycles, with the genomic data from each generation used to refine the predictive algorithms and improve the efficiency of the breeding program. The method demonstrates potential for accelerating genetic gain in livestock breeding while reducing the number of animals needed in the breeding program.

The foregoing general description is exemplary and explanatory only, and not restrictive of the disclosure. Other embodiments may be apparent to those skilled in the art from consideration of the specification and practice of the invention disclosed herein. It is intended that the specification and examples be considered as exemplary only.

1. A method for analyzing a bicephalic sperm cell, the method comprising:
receiving a sample comprising sperm cells;
isolating a bicephalic sperm cell from the sample, wherein the isolated bicephalic sperm cell comprises a first head and a second head;
separating the first head of the isolated bicephalic sperm cell while retaining the second head on the isolated bicephalic sperm cell;
performing, or causing to be performed, genomic analysis on the separated first head to produce genomic data; and
associating the genomic data with the isolated bicephalic sperm cell.

2. The method of claim 1, wherein isolating the bicephalic sperm cell comprises separating the sperm cells using a centrifugal device and isolating the bicephalic sperm cell based on a predetermined weight or a predetermined weight range.

3. The method of claim 2, wherein the predetermined weight is greater than or equal to about 20 pg.

4. The method of claim 1, wherein isolating the bicephalic sperm cell comprises separating the sperm cells using a flow cytometer and isolating the bicephalic sperm cell based on a predetermined optical property or a predetermined set of optical properties.

5. The method of claim 1, wherein the isolated bicephalic sperm cell is viable after separating.

6. The method of claim 1, wherein associating the genomic data with the isolated bicephalic sperm cell further comprises:
determining a structural or morphological feature associated with the isolated bicephalic sperm cell; and
based on the structural or morphological feature, determining a correspondence between the first head and the second head of the isolated bicephalic sperm cell.

7. The method of claim 1, wherein associating the genomic data with the isolated bicephalic sperm cell further comprises:
generating a prediction of an association of the genomic data with the isolated bicephalic sperm cell based on recombination hotspots, linkage disequilibrium patterns, and/or population-specific genetic variations.

8. The method of claim 1, wherein associating the genomic data with the isolated bicephalic sperm cell is performed by a machine learning system trained with annotated data associating genomic data of both heads of bicephalic sperm cells.

9. The method of claim 1, wherein associating the genomic data with the isolated bicephalic sperm cell is performed by a machine learning system trained with high-resolution images of sperm heads and associated genomic data of the sperm heads to detect structural or morphological features.

10. The method of claim 1, wherein associating the genomic data with the isolated bicephalic sperm cell is performed by a machine learning system trained to determine whether the first head and the second head derive from a single primary spermatocyte.

11. The method of claim 1, wherein separating the first head from the isolated bicephalic sperm cell comprises sequential compartmentalization of the separated first head and the isolated bicephalic sperm cell.

12. The method of claim 1, wherein the isolated bicephalic sperm cell is selected to fertilize an oocyte based on a determination that the isolated bicephalic sperm cell negates an aneuploidy condition.

13. The method of claim 11, further comprising, upon determining that the isolated bicephalic sperm cell has been used to fertilize the oocyte to generate an embryo, determining an inbreeding coefficient of the embryo via embryonic biopsy.

14. The method of claim 1, wherein associating the genomic data with the isolated bicephalic sperm cell comprises utilizing DNA barcodes to identify and track the isolated bicephalic sperm cell and the cleaved head.

15. The method of claim 1, further comprising, after separating the first head of the isolated bicephalic sperm cell:
cryopreserving the isolated bicephalic sperm cell while maintaining viability.

16. The method of claim 1, wherein separating the first head of the isolated bicephalic sperm cell while retaining the second head on the isolated bicephalic sperm cell comprises utilizing a microfluidics device.

17. The method of claim 1, wherein performing, or causing to be performed, genomic analysis on the separated first head to produce genomic data comprises performing probabilistic genomic prediction.

18. A system for analyzing a bicephalic sperm cell, comprising:
a flow cytometer configured to isolate the bicephalic sperm cell based on a predetermined optical property or a predetermined set of optical properties;
a microfluidics device integrated with the flow cytometer and configured to receive the isolated bicephalic sperm cell, wherein the microfluidics device is further configured to cleave a first head from the isolated bicephalic sperm cell while retaining the second head on the cell;
a sequence analyzer configured to receive the cleaved first head from the microfluidics device and perform genomic analysis to produce genomic data; and
a processor configured to associate the genomic data with the isolated sperm cell.

19. A method for enhanced assisted reproduction, the method comprising:
receiving a sample comprising sperm cells;
isolating a bicephalic sperm cell from the sample, wherein the isolated bicephalic sperm cell comprises a first head and a second head;
separating the first head of the isolated bicephalic sperm cell while retaining the second head on the isolated bicephalic sperm cell;
performing, or causing to be performed, genomic analysis on the separated first head to produce genomic data;
associating the genomic data with the isolated bicephalic sperm cell; and
selecting the isolated bicephalic sperm cell for in vitro fertilization, based on determining a probability that the isolated bicephalic sperm cell does not possess a chromosomal abnormality.

20. A method for enhanced animal breeding, the method comprising:
receiving a sample comprising sperm cells;
isolating a bicephalic sperm cell from the sample, wherein the isolated bicephalic sperm cell comprises a first head and a second head;
separating the first head of the isolated bicephalic sperm cell while retaining the second head on the isolated bicephalic sperm cell;
performing, or causing to be performed, genomic analysis on the separated first head to produce genomic data;
associating the genomic data with the isolated bicephalic sperm cell; and
selecting the isolated bicephalic sperm cell for in vitro fertilization, based on determining a probability that the isolated sperm cell possesses genetic material associated with desirable traits and a probability that the isolated sperm cell does not possess genetic material associated with undesirable traits.
